(19) United States
(12) Patent Application Publication
(10) Pub. No.: US 2008/0075657 A1
(43) Pub. Date: Mar. 27, 2008

(54) BIOPOLYMER SYSTEM FOR TISSUE SEALING

(76) Inventors: John M. Abrahams, Scarsdale, NY (US); Weiliam Chen, Mount Sinai, NY (US); Peter Zahos, Weston, FL (US)

Correspondence Address: SCHWEGMAN, LUNDBERG & WOESSNER, P.A., P.O. BOX 2938, MINNEAPOLIS, MN, 55402, US

(21) Appl. No.: 11/830,125

(22) Filed: Jul. 30, 2007

Related U.S. Application Data
(63) Continuation-in-part of application No. 11/530,362, filed on Sep. 8, 2006.
(63) Continuation-in-part of application No. 11/379,182, filed on Apr. 18, 2006.

(51) Int. Cl. A61K 51/00; A61K 38/18; A61K 9/14; A61K 48/00

(52) U.S. Cl. 424/1.11; 424/488; 514/12; 514/44

(57) ABSTRACT

The invention provides a method of treating degenerative disc disease and discogenic pain by disposing a hydrogel tissue sealant within the intervertebral disc, where the hydrogel fills voids and tears in the annulus fibrosus and replaces leaked material from the nucleus pulposus. The hydrogel is formed in situ from a substantially liquid premix, which can be emplaced with a syringe needle or a catheter into the intervertebral disc, where it forms the hydrogel by gelation. The hydrogel can also include a therapeutic or a protective material, or a radiopaque or MRI-active agent to aid in visualization.

### CLAIM OF PRIORITY FROM A PRIOR-FILED APPLICATION

This application claims priority to and is a Continuation-in-Part of U.S. patent application Ser. No. 11/379,182, filed Apr. 18, 2006, and U.S. patent application Ser. No. 11/530,362, filed Sep. 8, 2006. These applications are incorporated herein by reference in their entireties.

### FIELD OF THE INVENTION

The invention relates to the use of tissue sealants derived from biopolymers for the treatment of degenerative disc disease and discogenic pain.

### BACKGROUND OF THE INVENTION

Tissue Sealants and Hydrogels

Tissue sealants are increasingly important adjuncts in surgical procedures, being used in fields such as vascular surgery, cardiac surgery, spine surgery and brain surgery as well as in general surgery. Uses for tissue sealants include, among others, augmenting or replacing sutures to join tissues or place them in proximity, closing perforations in biological membranes to prevent leakage of fluids, incorporating medicinal substances at the location of emplacement for localized release, and filling areas of tissue removal. One commonly used tissue sealant is fibrin glue, a material analogous to clotted blood, which is obtained from the reaction of fibrinogen and thrombin isolated from blood plasma. For example, see “Fibrin Glue from Stored Human Plasma: An Inexpensive and Efficient Method for Local Blood Bank Preparation,” William D. Spotnitz, M. D., Paul D. Mintz, M. D., Nancy Avery, M. T., Thomas C. Bithell, M. D., Sanjiv Kaul, M. D., Stanton P. Nolan, M. D. (1987), The American Surgeon, 53, 460-62. However, concern about possible viral or prion contamination of human blood-derived protein products, and dissatisfaction with the relatively long times often required for fibrin gelation or “setting” to occur, have resulted in a search for tissue sealants with more advantageous properties.

There have been systems developed that use fibrin glues as part of a more complex assembly with more favorable properties. U.S. Pat. No. 6,699,484 discusses the use of fibrinogen in mixtures with polysaccharides such as hyaluronan and chitosan to form surgical adhesives, wherein the fibrinogen and thrombin components are distributed in dry form on a support comprising the polysaccharide, which is activated by water when emplaced on a wound to form a sealant.

In an attempt to avoid the use of human blood products, other mammalian sources of proteins have been studied. A tissue sealant has been prepared using bovine serum albumin that is crosslinked with glutaraldehyde. An example is BioGlue Surgical Adhesives produced by CryoLife, Inc. of Kennesaw, Georgia. However, bovine tissues are also a source of concern in terms of the possible presence of pathogenic entities such as viruses or prions. The types of processing required to destroy viruses or prions also tend to denature the desired proteins and make them intractable as sealants.

A tissue sealant that does not use proteins isolated from mammalian blood, such as Duraseal® produced by Confluent Surgical Inc. of Waltham, Mass., comprises tri-lysine-amine and an activated polyethyleneglycol. A similar product, termed CoSeal® and produced by Baxter of Deerfield, Ill., is likewise composed of synthetic functionalized polyethyleneglycol derivatives, also avoiding the use of blood-derived materials. However, both of these synthetic hydrogels are dimensionally unstable in the presence of water, undergoing considerable swelling. For example, see “Evaluation of Absorbable Surgical Sealants: In vitro Testing,” Patrick K. Campbell, PhD, Steven L. Bennett, PhD, Art Driscoll, and Amar S. Sawhney, PhD, at www.duralsealant.com/duralsealant/literature.htm (as of Aug. 24, 2006). This tendency to swell can be highly disadvantageous in certain applications, such as neurosurgery, where the resulting pressure on nerve or brain tissue can produce serious side-effects.

Chitin, a biopolymer that is abundant in the shells of arthropods, is a β-1,4 polymer of 2-acetamido-2-deoxyglucose. During its isolation, it is freed from proteinaceous and mineral components of the shell. Purified chitin can be further processed by chemical treatment resulting in deacetylation to yield chitosan, (poly-(2-amino-2-deoxyglucose)), which is a basic (alkaline) substance due to its free amino groups. From the perspective of medical uses, chitosan offers several desirable properties. The material is known to be non-toxic and biocompatible, and since chitin is not derived from vertebrates and is processed under rather harsh conditions such as exposure to alkalai during its transformation into chitosan, the possibility of contamination with viruses or prions that are pathogenic to mammals is very low. The utility of biocompatible chitosan derivatives in medical applications has received attention. For example, U.S. Pat. No. 5,093,319 discusses the use of films prepared from carboxymethylated chitosan for use in surgery to prevent post-operative adhesion of injured soft tissues upon healing. The chitosan derivatives are described to be formed into a biodegradable “sheet” that during surgery is emplaced between soft tissues for which adherence during healing is not desired. In another type of use, U.S. Pat. No. 4,532,134 discusses the use of chitosan in promoting blood coagulation in wounds.

Hydrogels are gels in which water is the dispersion medium. A common example of a hydrogel is a gel formed from the protein gelatin in water. Other hydrogels are formed by polysaccharides such as agar dispersed in water. Hydrogels in the form of sheets are used as wound dressings, where they are favored for their ability to help maintain a moist environment to facilitate healing of the wound without drying and cracking of tissues. For example, see www.medicaledu.com/hydrogellsheet.htm. Chemical derivatives of chitosan have also been used to form hydrogels for use as surgical sealants and in drug delivery devices. U.S. Pat. No. 6,602,952, assigned to Shearwater Corp., describes the preparation of poly(alkyleneoxide)chitosan derivatives and their use in the formation of hydrogels. The addition of these hydrophilic but non-ionic groups to the chitosan molecule alters its physical properties. Poly(alkyleneoxides) such as poly(ethyleneoxide), also known (somewhat inaccurately) as poly-ethyleneglycols or PEGs, are formed by the polymerization of alkylene oxides (epoxides) such as ethylene oxide. They may be obtained in a wide variety of molecular weights, with various structural features such as activated end groups, hydrolysable linkages, and others. For example, see the Nektar PEG catalog that lists a wide variety of the Shearwater functionalized PEGs, at www.nektar.com/pdf/nektar_catalog.pdf (as of Aug. 24, 2006).

Other methods have been described for the preparation of hydrogels from chitosan. The published PCT application WO2005/113608 and the published U.S. patent application no. 2005/0271729, both by the same inventor, discuss the crosslinking of chitosan and hyaluronan, also known as hyaluronic acid. Hyaluronan is an acidic linear polysaccharide formed of β-1,3 linked dimeric units, the dimeric units consisting of an 2-acetamido-2-deoxyglucose and D-gluconic acid linked in a β-1,4 configuration. These published applications discuss crosslinking the two types of polysaccharides using a carbodiimide reagent.

Hydrogels comprising chitosan derivatives and polybasic carboxylic acids or oxidized polysaccharides, for use in vascular occlusion, are also disclosed in copending U.S. patent application Ser. No. 11/425,280, filed Jun. 20, 2006 by the same inventors as in the present application.

Degenerative Disc Disease and Discogenic Back Pain

Low back pain is a debilitating health problem with the number of patients affected increasing on a yearly basis. Approximately 5.7 million people in the U.S. are diagnosed with discogenic back pain (DBP) each year (American Academy of Orthopaedic Surgeons). Discogenic Back pain is the most common cause of back pain.¹

Approximately 80% of the U.S. population will experience DBP at some point in their lives, 31 million Americans having discogenic lower back pain at any given time. Nearly 6 million new patients generate 15 million office visits each year, and the treatment cost of lower back pain exceeds $25 billion annually. The total societal cost of the disease, including lost work time, is estimated to exceed $85 billion per year.

The current standard treatment of DBP is spinal fusion. Pain associated with DBP is thought to occur due to transient hypermobility (excessive motion) in a given motion segment as a result of loss of disc height. Temporary pain relief is achieved through spinal fusion, which stiffens the affected motion segment. However, the stiffening of one motion segment as a result of spinal fusion, inevitably leads to hypermobility in adjacent segments causing adjacent segment degeneration (ASD), propagation of the disease along the spine, and the return of segmental pain.

The spinal disc consists of a gelatinous inner core (nucleus pulposus, NP) and an outer core (annulus fibrosus, AF). Patients with the clinical symptoms of Degenerative Disc Disease (DDD) generally undergo imaging of the lumbar spine during the course of their workup. Magnetic resonance imaging (MRI) is the most unequivocal method for documenting intervertebral disc pathology. The signal characteristics of the disc in T2-weighted images reflect changes caused by aging and degeneration. The signal loss of the disc on T2-weighed MRIs have been shown to correlate directly with the proteoglycan concentration.²

There is an association between the occurrence of low back pain and DDD; however the mechanism of DDD is not well understood. It is believed that an important factor in the pathogenesis of DDD is a decline in proteoglycans, which are normally very abundant in the disc nucleus. There is also an association between proteoglycan loss and the presence of inflammatory cytokines and mediators. It is known that degenerated and herniated discs have elevated levels of nitric oxide synthase (NOS), interleukin-6 (IL-6), prostaglandin E2 (PGE2) and matrix metalloproteases (MMPs), which are key biochemical mediators of the inflammatory cascade in degenerated discs.³

Progression of intervertebral disc disease causes the outer AF to lose its normal lamellar arrangement. As the disc fails, tissue fissures and clefts progress from the inner AF outward, towards the innervated outer layer, and contribute to the loss of mechanical integrity.⁴ The process of wound healing in normal tissues is from its depths to its superficial layers. However, in disc tissue, the process of healing is reverse in direction. The repair response to an annular tear begins in the vascularized outer layer. But when the tear occurs in the inner AF, the process of wound healing cannot be initiated.⁶ Inflammatory cytokines produced within the disc stimulate ingrowth of neural and vascular elements that are involved in the etiology of DBP. Additionally, diffusion of these inflammatory mediators from the NP along the fissures of the inner AF towards the innervated, vascularized outer AF and spinal nerve roots can contribute to DBP.⁷

Clinical interventions for DBP have largely focused on addressing the symptoms manifested (i.e. back pain) rather than directing treatments towards the fundamental causes of the problem. Degenerative Disc Disease (DDD) is the common factor causing DBP. Common surgical procedures performed on 15-20% of DDD patients include discectomy and laminectomy. For 2-3% of the population with DDD, spinal fusion surgery appears to be the best option to reduce pain. Although spinal fusion remains the standard, it is a highly invasive option, reserved as the last resort for severe back pain.

Patients are typically treated with a trial of anti-inflammatory agents, physical therapy with or without traction, and sometimes interventional procedures. Interventional procedures include discograms, epidural nerve root injections, and facet blocks. If the pain is due purely to discogenic disease without a disc herniation, patients will undergo a discogram to “diagnose” the worst level. A discogram works by injecting a needle into the lumbar disc space and measuring intradiscal pressure. Saline is infused into the disc space and any reproduction of pain is noted.

Intervertebral disc degeneration is a common occurrence during adult life that has adverse economic consequences on the health care system. Current surgical treatments are aimed at removing or replacing the degenerate tissue, which can alter the biomechanics of the spine and result in degeneration at adjacent disc levels. The ideal treatment of the degenerate disc would involve biologic repair, and tissue-engineering techniques offer a means to achieve this goal.

Alini et al used scaffolds of type I collagen and hyaluronan seeded with bovine nucleus pulposus or anulus fibrosus cells and maintained culture for up to 60 days. During the culture period, various proteoglycans (aggrecan, decorin, biglycan, fibromodulin, and lumican) and collagens (types I and II) accumulated in the scaffold. Their work demonstrated that although it is possible to maintain functional disc cells in a biomatrix, it is necessary to optimize proteoglycan synthesis and retention if any resulting tissue is to be of value in the biologic repair of the degenerate disc. The same group then showed that cationic chitosan could form an ideal environment in which large quantities of newly synthesized anionic proteoglycan could be entrapped (Roughley). Their in vitro results supported the concept that chitosan may be a suitable scaffold for cell-based supplementation to help restore the function of the NP during the early stages of disc degeneration.

Other groups have investigated the use of a modified chitosan complexed with hydroxybutyl groups to deliver stem cells and gelate in the disc space (Dang). Dang et al showed the potential of hydroxylbutyl chitosan gel as an injectable carrier for future applications of delivering therapeutics to encourage a biologically relevant reconstruction of the degenerated disk. Mwale et al investigated the gelation kinetics of various concentrations of two water-soluble chitosan chlorides and two chitosan glutamates. Results showed that when injected into the degenerated nucleus pulposus of human cadaveric intervertebral disk, the gel flowed into the clefts without leakage and was thought to be a promising agent for tissue engineering and repair of the intervertebral disc.

In Pfeiffer et al. prospectively, with randomized segment-treatment assignment, and with blinded evaluators, lumbar motion segments in Cercopithecus monkeys were analyzed for macroscopic and radiological changes 24 weeks after nucleotomy and nucleotomy with additional intradiscal application of different hyaluronic acid formulations versus untreated control segments. The objective was to find out whether hyaluronic acid is able to influence the degenerative cascade in nonhuman primates after nucleotomy. In a similar procedure, hyaluronic acid has proven to decrease degeneration after nucleotomy in a Minipig model. Segments with high-molecular-weight hyaluronic acid (Hylan G-F 20) application proved to be significantly superior over those with a standard nucleotomy in radiographs, MR images, CT scans, and macroscopic appearance at follow-up.

### REFERENCES

1. Praemer A, Furner S, Rice D P. Musculoskeletal Conditions in the United States. Rosemont, Ill.: American Academy of Orthopaedic Surgeons; 1999.
2. Pfirrmann C W, Metzdorf A, Zanetti M, Hodler J, Boos N “Magnetic resonance classification of lumbar intervertebral disc degeneration.” Spine 26(17):1873-8, 2001.
3. Wisnecki, in Rothman et al, The Spine, W.B. Saunders Company, 1992.
4. Peng, B, Hao, J, Hou, S, Wu, W, Jiang, D, Fu, X, Yang, Y. “Possible pathogenesis of painful intervertebral disc degeneration.” Spine, 31(5):560-566, 2006.
5. Di Martino A, Vaccaro A R, Lee J Y, Denaro V, Lim M R. “Nucleus pulposus replacement.” Spine 30(16S):S16-S22, 2005.
6. Domish M, Kaplan D, Skaugrud 0. “Standards and guidelines for biopolymers in tissue-engineered medical products.” Ann. NY Acad, Sci, 944:388-397, 2001
7. Kang J D, Stefanovic-Racic M, McIntyre L A, Georgescu H I, Evans C H. “Toward a biochemical understanding of human intervertebral disc degeneration and herniation. Contributions of nitric oxide, interleukins, prostaglandin E2, and matrix metalloproteinases.” Spine, 22(10), 1065-1073, 1997.
8. Roughley P, Hoemann C, DesRosiers E, Mwale F, Antoniou J, Alini M: The potential of chitosan-based gels containing intervertebral disc cells for nucleus pulposus supplementation. Biomaterials: 27(3):388-96, 2006.
9. Alini M, Li W, Markovic P, Aebi M, Spiro R C, Roughley P J. The potential and limitations of a cell-seeded collagen/hyaluronan scaffold to engineer an intervertebral disc-like matrix. Spine: 28(5):446-54, 2003.
10. Dang J M, Sun D D, Shin-Ya Y, Sieber A N, Kostuik J P, Leong K W: Temperature-responsive hydroxybutyl chitosan for the culture of mesenchymal stem cells and intervertebral disk cells. Biomaterials 27(3):406-18, 2006.
11. Mwale F, lordanova M, Demers C N, Steffen T, Roughley P, Antoniou J. Biological evaluation of chitosan salts cross-linked to genipin as a cell scaffold for disk tissue engineering. Tissue Eng 11 (1-2):130-40, 2005.
12. Pfeiffer M, Boudriot U, Pfeiffer D, Ishaque N, Goetz W, Wilke A. Intradiscal application of hyaluronic acid in the non-human primate lumbar spine: radiological results. Eur Spine J 12(1):76-83, 2003.

Thus, there is an ongoing need for a hydrogel tissue sealant that is not blood or animal protein derived, that consists of biocompatible materials, is dimensionally stable after emplacement in the patient's body, has good sealant and tissue adhesive properties, is of sufficient strength and elasticity to effectively seal biological tissues, that can be readily prepared and used during surgery, that forms the tissue seal on a timescale compatible with surgery on living patients, and that can be used for the repair of vertebral discs and the treatment of discogenic pain.

### SUMMARY OF THE INVENTION

The present invention concerns a method of treatment of degenerative disc disease or of discogenic pain, comprising forming in situ within a tear void of an intervertebral disc a hydrogel, the hydrogel being formed by gelation of a premix, the premix including an alkylated chitosan and an oxidized polysaccharide. The alkylated chitosan can be a PEG-chitosan or an acrylated chitosan. The oxidized polysaccharide can be an oxidized dextran or an oxidized starch. The premix can also include an acidic polysaccharide, for example, hyaluronan.

An embodiment of the inventive method is directed to the use of a hydrogel tissue sealant that, due to its exceptional dimensional stability, may be used in situations where swelling and the resulting pressure are undesirable and produce unwanted side effects.

An embodiment of the inventive method is directed to the use of a hydrogel tissue sealant that offers a very low risk of contamination by pathogens such as viruses and prions.

An embodiment of the inventive method further provides for the use in vertebral disc repair of a tissue sealant that is not prepared from human blood products, which is desirable because human blood products carry a risk of contamination with pathogens and are also objectionable to certain patients on religious and moral grounds.

An embodiment of the inventive method provides for use in vertebral disc repair a composition that includes a chitosan derivative that has been modified by the introduction of covalently bound moieties onto the polymer chain. The chitosan derivative, and an oxidized polysaccharide, and optionally an acidic polysaccharide, upon dissolution in an aqueous medium can initially form a flowable, substantially liquid sol, a premix, that over a period of time, typically in the order of minutes, gels to form a hydrogel adapted for use in the method of the invention. The hydrogel, which is biocompatible and can be biodegradable, when formed in situ serves to fill and seal annular voids in vertebral discs and to replace lost nucleus pulposus material that has leaked out of a herniated disc. By this repair and through incorporation of therapeutic and protective agents in the hydrogel, degenerative disc disease is treated and discogenic pain alleviated.

In another embodiment of the inventive method of using of the hydrogel for disc repair, the hydrogel may further comprise a protective or therapeutic material or substance. The substance may be an antibiotic, an anticancer agent, a peptide, a protein, a nucleic acid or a nucleic acid analog, a radioactive material, a recombinant protein, a growth factor, a plurality of stem cells, or an anti-inflammatory agent such as indomethacin, a steroid, an interleukin, vascular endothelial growth factor, or a cytokine, or any combination thereof, where it is advantageous to provide the substance within the vertebral disc where the hydrogel is emplaced.

For example, the protein may be a growth factor, such as a vascular growth factor or a factor that induces a particular kind of tissue growth. In another specific embodiment, the protein may be an inhibitory factor, such as a receptor antagonist such as for a growth factor, when supply of an inhibitory factor is desirable.

In yet another specific embodiment, the nucleic acid may be an antisense nucleic acid, or a small interfering nucleic acid analog, wherein it is advantageous to securely emplace the material for treatment of a condition responsive to such therapy.

In another embodiment, the therapeutic agent may be an antibiotic to inhibit bacterial infection. Or, a protective agent may be an anti-inflammatory substance wherein it is advantageous to supply the substance directly at the site of damage that is repaired with the tissue sealant, such as to reduce swelling and resulting pressure on surrounding tissues.

In another embodiment, the hydrogel comprises a dye, such as a visible dye or a radio-opaque dye, to enable visualization of the position of localization of the hydrogel in the disc. Alternatively, the hydrogel can include an MRI-active agent to enable visualization of the disposition of the hydrogel using MI techniques. The agents can all be introduced by the expedient of including them within the premix prior to emplacement within the vertebral disc.

In another embodiment, the hydrogel comprises a microsphere or a nanosphere, preferably a large number of microspheres or nanospheres dispersed in the hydrogel. Preferably the microsphere or nanosphere contains a therapeutic agent or a protective agent. The plurality of microspheres or nanospheres can be introduced into the hydrogel by inclusion within the premix prior to emplacement.

The invention further provides a kit adapted for preparing and using the tissue sealant of the invention, the kit comprising a first container and a second container, wherein the first container comprises an alkylated chitosan, the first container further comprising an aqueous medium or being adapted for addition thereto of an aqueous medium; and the second container comprising an oxidized polysaccharide, the second container further comprising an aqueous medium or being adapted for addition thereto of an aqueous medium. Optionally, an acidic polysaccharide can be included. The kit can contain instructional material. The kit can also contain a mixing apparatus for the contents of the two containers, such as a pair of coupled syringes wherein the alkylated chitosan and the oxidized polysaccharide, and optionally the acidic polysaccharide, can be mixed in an aqueous medium to prepare a premix suitable for emplacement within the vertebral disc according to the inventive method. The kit can also include a syringe needle or a catheter adapted for introduction of the premix into the damaged vertebral disc.

### DETAILED DESCRIPTION OF THE INVENTION

### Definitions

As used herein a “disc” refers to an intervertebral disc, the elastic structure disposed between adjacent vertebrae that provides cushioning between the vertebrae and serves to hold them together. The disc includes two major structures, the gelatinous inner core, the nucleus pulposus (NP), and a fibrous ring-shaped structure surrounding the NP, the annulus fibrosus (AF). The annulus fibrosus consists of several layers of fibrocartilage, which is composed of fibrous connective tissue disposed in bundles and surrounded by cartilage. The annular fibers contain the nucleus pulposus and assist in distributing pressure evenly across the disc. The nucleus pulposus contains loose fibers suspended in a mucoprotein gel.

An “annular void” is a void in the annulus fibrosus resulting from a tear of the tissue, which can be a result of aging of the tissue or trauma. When the nucleus pulposus material begins to exude through a tear, this is referred to as a “herniated disc.” A decreasing ability of the nucleus pulposus to provide cushioning, and damage to the annulus fibrosus, can result in “axial” or “discogenic” pain. When the exuding nucleus pulposus material puts pressure on a nearby nerve, a painful condition results which is known as “sciatica.” A sensation of pain can also result from the contact of inflammatory proteins contained within the nucleus pulposus gel with a nerve. Dehydration and degeneration of the nucleus pulposus is part of the syndrome of “degenerative disc disease.”

As used herein, the term “to seal” or “sealing” refers to the act wherein two physically noncontiguous tissues or portions thereof are joined together, or where a hole, tear, cut, perforation or other discontinuity is repaired so as to close the hole, tear, cut or perforation. Sealing implies at least some degree of adhesion of the material used to the tissue to which it is applied, such that the sealed tissue is secured against at least a moderate displacing force. The discontinuity in the tissue that is being sealed may be an incision made as part of a surgical procedure, or it may be a wound. A “sealant” is a material which is used to seal tissue. As mentioned, a sealant adheres, at least to some degree, to the tissue which is being sealed, such that the sealant material is unlikely in the short term to detach from the repaired or sealed tissue under the influence of at least a moderate force, such as may be experienced when a patient to whom the sealant has been applied moves in a normal fashion. The sealant may be biodegradable and eventually dissolve or be absorbed into the patient's body without departing from the principles of the invention.

The degree of force that is needed to disrupt a seal formed according to the invention may vary. If the tissue is “sealed,” as the term is used herein, the degree of adhesivity may be relatively low, such that the sealant serves to fill a void in the tissue or to keep the tissue portions in conjunction when they are not subject to a high degree of strain. If a tissue is “adhesively sealed,” as the term is used herein, a higher degree of strain is necessary to disrupt the bond between the tissue portions, such that rupture of the seal only takes place when a relatively high degree of force is applied. Thus, a tissue may be sealed such that the joined tissue portions are held in proximity by a sealant but application of a relatively high degree of strain would tend to separate the portions and rupture the seal, or the tissue may be adhesively sealed such that a relatively high degree of strain is needed to rupture the seal.

“Adhere” or “adherence” refers to the creation of a physical bond between the material and tissue such that a moderate motion or force does not cause separation of the material from the tissue on which it is disposed. Thus, a tissue sealant serves to glue together living tissue, at least temporarily, such as for the amount of time it takes healing to occur. However, sealing may take place for a more prolonged period without departing from the principles of the invention. The physical bond that is created between the material and the tissue that is being sealed may have one or several bases including electrostatic bonding and covalent bonding, but any mechanism by which the adherence takes place falls within the definition herein.

The terms “adhesive” and “adhesivity” similarly refer to the existence of a physical bond between two materials such as a tissue sealant and the tissue to which the sealant is applied. An adhesive is a material which adheres to tissue or other material and which may be used to constrain the separation of two tissue masses. Adhesivity is the property or degree to which a material adheres to a tissue or other material. As used herein, adhesive tissue sealants are those sealants of the invention that are adapted to hold the tissue portions being sealed together against a relatively high degree of rupturing strain.

As used herein, a “hydrogel” refers to a material of solid or semi-solid texture that includes water. Hydrogels are formed by a three-dimensional network of molecular structures within which water, among other substances, may be held. The three-dimensional molecular network may be held together by covalent chemical bonds, or by ionic bonds, or by any combination thereof. A common example of a hydrogel is gelatin, a protein, that “sets up” or forms a gel from a sol upon heating and subsequent cooling. Not all substances that form hydrogels are proteins; polysaccharides such as starches may also form hydrogels. Still other hydrogels may be formed through the mixture of two or more materials that undergo chemical or physical reactions with each other to create the three-dimensional molecular network that provides the hydrogel with a degree of dimensional stability. Such mixtures of materials that interact or react with each other to form a hydrogel are referred to herein as a “premix.” Thus, a “premix” as used herein refers to a mixture of materials that after mixing will gel, or “set up,” to form the hydrogel. A premix can be of a liquid or semi-liquid texture such that it can be pumped or transferred by the methods usually used for liquids, such as flow through tubes such as syringe needles or catheters.

The act of “gelation” refers to the formation of a gel from a sol. In some cases, the sol may consist of a single material dispersed in a solvent, typically water, as in the case of gelatin. In other cases, the sol may consist of more than a single material dispersed in a solvent wherein the several materials will eventually react with each other to form a gel, and when the solvent in which they are dispersed comprises water, the gel is a hydrogel. The hydrogels disclosed and claimed herein are of the type that are formed by the mixture of more than a single component.

A “saccharide” as used herein refers to a carbohydrate. The term “carbohydrate” includes the class of compounds commonly known as sugars, in addition to compounds that are chemically related to sugars. The term thus includes simple “monosaccharide” sugars, “disaccharide” sugars, as well as polymeric “polysaccharides.” The term encompasses a group of compounds including sugars, starches, gums, cellulose and hemicelluloses. The term further encompasses sugar derivatives such as amino-sugars, for example, 2-amino-2-deoxyglucose, as well as their oligomers and polymers; sulfated sugars; and sugars with hydroxyl, amino, carboxyl and other groups. A polysaccharide can be homogeneous, formed from just a single type of monosaccharide unit. An example is cellulose, formed solely of β-D-glycose units. A polysaccharide can also be heterogeneous, formed from more than one type of monosaccharide unit. An example is hyaluronic acid, also known as hyaluronan, which is formed of alternating glucuronic acid and N-acetyl glucosamine units.

A carbohydrate as defined herein comprises sugars or sugar derivatives with beta (β) or alpha (α) anomeric stereochemistry; moreover, the sugars can have (R) or (S) relative configurations, can exist as the (+) or (−) isomer, and can exist in the D or L configuration. The terms “anomer” and “anomeric” refer to the stereochemical configuration at the acetal, hemiacetal, or ketal carbon atom, as is well known in the art.

As used herein, “chitosan” refers to a polysaccharide polymer, either obtained from a natural source such as chitin, or synthetically prepared. Chemically, chitosan is predominantly a polymer of β-1,4-linked 2-amino-2-deoxyglucose monomers. When prepared from a natural source, the usual natural source is chitin, a major constituent of the shells of crabs, shrimp and other arthropods. Chitin is chemically a polymer comprising β-1,4-linked 2-acetamino-2-deoxyglucose monomers. After isolation of chitin from its natural source, it is treated in a manner as to cause hydrolysis of the acetamido group without cleavage of the sugar-sugar bonds, typically through alkaline hydrolysis. Chitosan is not a single molecular entity, but comprises polymeric chains of various lengths.

As used herein, an “alkylated chitosan” is a material formed of chitosan molecules to which carbon-containing molecules have been bonded. The term “alkylated chitosan” thus comprises a large number of possible chemical structures, but they all share the unifying feature that chemical bonds have been formed between the components of the chitosan molecules and at least one carbon atom in each of the molecules that are bonded to the chitosan. Specific examples of alkylated chitosan within the meaning herein include poly(oxyalkylene)chitosan, wherein poly(oxyethylene), or polyethyleneglycol, chains are covalently bonded to the chitosan backbone, as well as acrylated chitosans, formed by alkylation of chitosan with acrylates, such as sodium acrylate.

When referring to the “molecular weight” of a polymeric species such as an alkylated chitosan, a weight-average molecular weight is being referred to herein, as is well known in the art.

A “degree of substitution” of a polymeric species refers to the ratio of the average number of substituent groups, for example an alkyl substituent, per monomeric unit of the polymer as defined.

A “degree of polymerization” of a polymeric species refers to the number of monomeric units in a given polymer molecule, or the average of such numbers for a set of polymer molecules.

A “poly(oxyalkylene)chitosan” is a variety of alkylated chitosan as defined herein. A “poly(oxyalkylene)” group is a polymeric chain of atoms wherein two carbon atoms, an ethylene group, are bonded at either end to oxygen atoms. The carbon atoms of the ethylene group may themselves bear additional radicals. For example, if each ethylene group bears a single methyl group, the resulting poly(oxyalkylene) group is a poly(oxypropylene) group. If the ethylene groups are unsubstituted, the poly(oxyalkylene) group is a poly(oxyethylene) group. A poly(oxyethylene) group may be of a wide range of lengths, or degrees of polymerization, but is of the general molecular formula of the structure [—CH₂—CH₂—O—CH₂—CH₂—O—]ₙ, where n may range from about 3 upwards to 10,000 or more. Commonly referred to as “polyethyleneglycol” or “PEG” derivatives, these polymeric chains are of a hydrophilic, or water-soluble, nature. Thus, a poly(oxyalkylene)chitosan is a chitosan derivative to which poly(oxyalkylene) groups are covalently attached. A terminal carbon atom of the poly(oxyalkylene) group forms a covalent bond with an atom of the chitosan chain, likely a nitrogen atom, although bonds to oxygen or even carbon atoms of the chitosan chain may exist. Poly(oxyethylene)chitosan is often referred to as “polyethyleneglycol-grafted chitosan” or “PEG-chitosan” or “PEG-g-chitosan” or “PEG-grafted-chitosan.”

The end of the poly(oxyethylene) chain that is not bonded to the chitosan backbone may be a free hydroxyl group, or may comprise a capping group such as methyl. Thus, “polyethylene glycol” or “poly(oxyethylene)” or “poly(oxyalkylene)” as used herein includes polymers of this class wherein one, but not both, of the terminal hydroxyl groups is capped, such as with a methyl group. In a specific method of preparation of the poly(oxyethylene)chitosan, use of a polyethyleneglycol capped at one end, such as MPEG (methyl polyethyleneglycol) may be advantageous in that if the PEG is first oxidized to provide a terminal aldehyde group, which is then used to alkylate the chitosan via a reductive amination method, blocking of one end of the PEG assures that no difunctional PEG that may crosslink two independent chitosan chains is present in the alkylation reaction. It is preferred to avoid crosslinking in preparation of the poly(oxyethylene)chitosans of the present invention.

An alkylated chitosan is also a chitosan to which other carbon-containing molecules are linked. An “acrylated chitosan” as the term is used herein is an alkylated chitosan wherein acrylates have been allowed to react with, and form chemical bonds to, the chitosan molecule. An acrylate is a molecule containing an α,β-unsaturated carbonyl group; thus, acrylic acid is prop-2-enoic acid. An acrylated chitosan is a chitosan wherein a reaction with acrylates has taken place. The acrylate may bond to the chitosan through a Michael addition of the chitosan nitrogen atoms with the acrylate.

As used herein, the term “acidic polysaccharide” refers to polymeric carbohydrates comprising carboxylic acid groups. The polymeric carbohydrate can be naturally occurring, or can be synthetic or semi-synthetic. Examples of acidic polysaccharides are hyaluronan (hyaluronic acid) and carboxymethyl cellulose. Carboxymethylcellulose, as is well-known in the art, is prepared by reaction of cellulose with sodium chloroacetate, and the product is believed to contain acidic carboxymethyl groups covalently linked to the primary hydroxyl groups of the anhydroglucose monomeric units that make up the cellulose molecule.

The term “oxidized polysaccharide” refers to a polysaccharide, acidic or non-acidic, that has undergone treatment with an oxidizing reagent, such as sodium periodate, that cleaves vicinal diol moieties of the carbohydrate to yield aldehyde groups. An oxidized hyaluronan, that is, hyaluronan that has been treated with an oxidizing agent, such as sodium periodate, that cleaves vicinal diol moieties and provides aldehyde groups, is an example of an oxidized polysaccharide as well as of an acidic polysaccharide within the meanings herein. An oxidized dextran, that is, dextran that has been treated with an oxidizing agent, such as sodium periodate, that cleaves vicinal diol moieties and provides aldehyde groups, is another example of an oxidized polysaccharide within the meaning herein. Oxidized dextran is a neutral oxidized polysaccharide in that it does not contain any substantial content of carboxylic acid groups. Another example of an oxidized polysaccharide is an oxidized starch, that is, a starch that has been treated with an oxidizing agent, such as sodium periodate, that provides aldehyde groups. Oxidized starch is also a neutral oxidized polysaccharide. It is believed that the aldehyde groups of oxidized polysaccharides interact with the amino groups of an alkylated chitosan in such a way as to markedly increase the viscosity of the mixture and cause gelation. While not wishing to be bound by theory, it is believed that this intermolecular interaction includes covalent bond formation and takes place through the formation of imines or Schiff bases, or alternatively of hemi-aminals, between the amino groups and the aldehyde groups.

An “aqueous medium,” as the term is used herein, refers to a liquid medium composed largely, but not necessarily exclusively, of water. Other components may also be present, such as salts, co-solvents, buffers, stabilizers, dispersants, colorants and the like.

As used herein, the act of “mixing between mutually coupled syringes” refers to a procedure wherein one syringe is partially filled with one ingredient, a second syringe is partially filled with a second ingredient, and the two syringes are coupled together as with a luer connector such that the contents of the syringes are mixed by drawing the contents of one syringe through the connector into the second syringe, then reciprocally expelling the contents of the second syringe back into the first syringe. This process may be repeated until adequate mixing is achieved.

A “therapeutic agent” is any agent which serves to repair damage to a living organism to heal the organism, to cure a malcondition, to combat an infection by a microorganism or a virus, or to assist the body of the living mammal to return to a healthy state. A “protective agent” is any agent which serves to prevent the occurrence of damage to an organism, such as by preventing the establishment of an infection by a microorganism, to prevent the establishment of a malcondition, or to preserve an otherwise healthy body in the state of health. Therapeutic and protective agents comprise pharmaceuticals, radiopharmaceuticals, hormones or their analogs, enzymes, materials for genetic therapy such as antisense nucleotides or their analogs, macroscopic ingredients such as bone powder as is used to induce bone growth, growth factors as may be used to stimulate tissue growth such as by angiogenesis, or any other such agents as are medically advantageous for use to treat a pathological condition. As used herein, “treating” or “treat” includes (i) preventing a pathologic condition from occurring (e.g. prophylaxis); (ii) inhibiting the pathologic condition or arresting its development; (iii) relieving the pathologic condition; and/or (iv) diminishing symptoms associated with the pathologic condition.

A therapeutic agent or a protective agent may comprise a “drug.” As used herein, a “drug” refers to a therapeutic agent or a diagnostic agent and includes any substance, other than food, used in the prevention, diagnosis, alleviation, treatment, or cure of a disease. Stedman's Medical Dictionary, 25^{th} Edition (1990). The drug can include any substance disclosed in at least one of: The Merck Index, 12^{th} Edition (1996); Pei-Show Juo, Concise Dictionary of Biomedicine and Molecular Biology, (1996); U.S. Pharmaconeia Dictionary, 2000 Edition; and Physician's Desk Reference, 2001 Edition.

Specifically, the drug can include, but is not limited to, one or more polynucleotides, polypeptides, oligonucleotides, gene therapy agents, nucleotide analogs, nucleoside analogs, polynucleic acid decoys, therapeutic antibodies, anti-inflammatory agents, blood modifiers, anti-platelet agents, anti-coagulation agents, immune suppressive agents, anti-neoplastic agents, anti-cancer agents, anti-cell proliferation agents, and nitric oxide releasing agents.

The polynucleotide can include deoxyribonucleic acid (DNA), ribonucleic acid (RNA), double stranded DNA, double stranded RNA, duplex DNA/RNA, antisense polynucleotides, functional RNA or a combination thereof. In one embodiment, the polynucleotide can be RNA. In another embodiment, the polynucleotide can be DNA. In another embodiment, the polynucleotide can be an antisense polynucleotide.

The polynucleotide can be a single-stranded polynucleotide or a double-stranded polynucleotide. The polynucleotide can have any suitable length. Specifically, the polynucleotide can be about 2 to about 5,000 nucleotides in length, inclusive; about 2 to about 1000 nucleotides in length, inclusive; about 2 to about 100 nucleotides in length, inclusive; or about 2 to about 10 nucleotides in length, inclusive.

An antisense polynucleotide is typically a polynucleotide that is complimentary to an mRNA, which encodes a target protein. For example, the mRNA can encode a cancer promoting protein i.e., the product of an oncogene. The antisense polynucleotide is complimentary to the single stranded mRNA and will form a duplex and thereby inhibit expression of the target gene, i.e., will inhibit expression of the oncogene. The antisense polynucleotides of the invention can form a duplex with the mRNA encoding a target protein and will disallow expression of the target protein.

A “gene therapy agent” refers to an agent that causes expression of a gene product in a target cell through introduction of a gene into the target cell followed by expression. An example of such a gene therapy agent would be a genetic construct that causes expression of a protein, such as insulin, when introduced into a cell. Alternatively, a gene therapy agent can decrease expression of a gene in a target cell. An example of such a gene therapy agent would be the introduction of a polynucleic acid segment into a cell that would integrate into a target gene and disrupt expression of the gene. Examples of such agents include viruses and polynucleotides that are able to disrupt a gene through homologous recombination. Methods of introducing and disrupting genes within cells are well known to those of skill in the art.

Nucleotide and nucleoside analogues are well known on the art. Examples of such nucleoside analogs include, but are not limited to, Cytovenee (Roche Laboratories), Epivir® (Glaxo Wellcome), Gemzar® (Lilly), Hivid® (Roche Laboratories), Rebetron® (Schering), Videx® (Bristol-Myers Squibb), Zerit® (Bristol-Myers Squibb), and Zovirax® (Glaxo Wellcome). See, Physician's Desk Reference, 2001 Edition.

As used herein, a “peptide” and a “protein” refer to polypeptides, linear polymers of amino acids, the difference between the terms “peptide” and “protein” largely being in the length of the polymer. In one embodiment, the polypeptide can be an antibody. Examples of such antibodies include single-chain antibodies, chimeric antibodies, monoclonal antibodies, polyclonal antibodies, antibody fragments, Fab fragments, IgA, IgG, IgM, IgD, IgE and humanized antibodies. In one embodiment, the antibody can bind to a cell adhesion molecule, such as a cadherin, integrin or selectin. In another embodiment, the antibody can bind to an extracellular matrix molecule, such as collagen, elastin, fibronectin or laminin. In still another embodiment, the antibody can bind to a receptor, such as an adrenergic receptor, B-cell receptor, complement receptor, cholinergic receptor, estrogen receptor, insulin receptor, low-density lipoprotein receptor, growth factor receptor or T-cell receptor. Antibodies of the invention can also bind to platelet aggregation factors (e.g., fibrinogen), cell proliferation factors (e.g., growth factors and cytokines), and blood clotting factors (e.g., fibrinogen). In another embodiment, an antibody can be conjugated to an active agent, such as a toxin or a radionuclide.

An “anti-cancer agent” means an agent that either inhibits the growth of cancerous cells, or causes the death of cancerous cells. Anti-cancer agents include, e.g., nucleotide and nucleoside analogs, such as 2-chloro-deoxyadenosine, adjunct antineoplastic agents, alkylating agents, nitrogen mustards, nitrosoureas, antibiotics, antimetabolites, hormonal agonists/antagonists, androgens, antiandrogens, antiestrogens, estrogen & nitrogen mustard combinations, gonadotropin releasing hotmone (GNRH) analogues, progestrins, immunomodulators, miscellaneous antineoplastics, photosensitizing agents, and skin & mucous membrane agents. See, Physician's Desk Reference, 2001 Edition.

An “antimicrobial,” as used herein, refers to a molecular entity that is effective as a therapeutic agent or as a protective agent against an infection by a microorganism, which could be a bacterium, a protozoan, a fungus, a virus, or another pathogenic living organism. An antimicrobial may be an antibiotic, effective against bacteria, including aminoglycoside antibiotics such as gentamicin or streptomycin, a cephalosporin such as cephalexin or cephtriaxone, a carbacephem such as loracarbef, a glycopeptide such as vancomycin, a macrolide such as erythromycin, a penicillin such as amoxicillin or ampicillin, a polypeptide such as bacitracin or polymyxin B, a quinolone such as ciprofloxacin, a tetracycline such as oxytetracycline, a sulfonamide, or any other medically approved agent for treatment of bacterial infections. Alternatively the antimicrobial may be an antifungal agent such as ketoconazole, miconazole or amphotericin B, or an antiviral agent such as acyclovir or AZT.

A “radioactive material” as used herein refers to any naturally occurring or manmade substance that emits ionizing radiation such as gamma rays, beta particles, Auger electrons, X-rays, or alpha particles. A radioactive material may be used for diagnostic purposes, such as for imaging as in positron emission tomography (PET). A radionuclide commonly used for imaging diagnostics is fluorine-18. Alternatively a radioactive material may be used for therapeutic purposes, as in treating tumors. Radionuclides used therapeutically include technetium-99m, iodine-123 and -131, and gallium-67, among others.

A “radiopaque” material or agent is an agent that interferes with the transmission of ionizing radiation, particularly X-rays that are used for medical imaging. When a material containing a radiopaque agent is disposed within the body of a patient and an X-ray or fluoroscopic image is obtained, the radiopaque agent serves to visualize the disposition of the material within the body. Examples of radiopaque agents include iodine-containing organic compounds, and metals such as gold, tantalum, and barium, either in elemental or in salt form. An “MRI-active” agent is an agent that alters the magnetic resonance response of a tissue or material containing the agent within the body of a patient, such that when an MRI image of the patient is obtained, the disposition of the material within the body can be visualized in the MRI image. An example of an MRI-active agent is a gadolinium salt.

In the claims provided herein, the steps specified to be taken in a claimed method or process may be carried out in any order without departing from the principles of the invention, except when a temporal or operational sequence is explicitly defined by claim language. Recitation in a claim to the effect that first a step is performed then several other steps are performed shall be taken to mean that the first step is performed before any of the other steps, but the other steps may be performed in any sequence unless a sequence is further specified within the other steps. For example, claim elements that recite “first A, then B, C, and D, and lastly E” shall be construed to mean step A must be first, step E must be last, but steps B, C, and D may be carried out in any sequence between steps A and E and the process of that sequence will still fall within the four corners of the claim.

Furthermore, in the claims provided herein, specified steps may be carried out concurrently unless explicit claim language requires that they be carried out separately or as parts of different processing operations. For example, a claimed step of doing X and a claimed step of doing Y may be conducted simultaneously within a single operation, and the resulting process will be covered by the claim. Thus, a step of doing X, a step of doing Y, and a step of doing Z may be conducted simultaneously within a single process step, or in two separate process steps, or in three separate process steps, and that process will still fall within the four corners of a claim that recites those three steps.

Similarly, except as explicitly required by claim language, a single substance or component may meet more than a single functional requirement, provided that the single substance fulfills the more than one functional requirement as specified by claim language.

### DETAILED DESCRIPTION

A hydrogel for use in treatment of degenerative disc disease and of discogenic pain according to the method of the present invention is a hydrogel that achieves a gelled state from a substantially liquid or semi-liquid premix after a period of time, typically in the order of minutes. The premix contains more than a single polymeric component. For example, the premix can include two polymeric components in an aqueous medium; an alkylated chitosan such as acrylated chitosan, and an oxidized polysaccharide such as oxidized dextran. Alternatively, the premix can include three polymeric components in an aqueous medium; an alkylated chitosan such as acrylated chitosan, an oxidized polysaccharide such as oxidized dextran, and an acidic polysaccharide such as hyaluronan.

The hydrogel, which can be used to repair the vertebral discs of a living mammal such as a human patient according to the inventive method, is formed upon gelation of the premix. Mixing of the components that make up the premix provides a liquid or semi-liquid sol that may be pumped or transferred by any technique suitable for handling somewhat viscous liquid materials, such as syringes, pipettes, tubing, catheters, and the like. Upon standing, such as after emplacement of the premix with a vertebral disc, the premix sol after a period of time gels or “sets up” into the hydrogel, in situ within the vertebral disc, thus filling and sealing voids and tears in the annulus fibrosus and replacing leaked nucleus pulposus materials. This serves to restore the resiliency of the disc, providing a normal level of vertebral support, and promoting healing of the damaged disc.

The premix sol and the resulting hydrogel that forms from the sol are suitable for contact with living biological tissue. Thus, the hydrogel can remain in contact with living biological tissue within a human patient for an extended period of time without damaging the tissue on which it is disposed. The hydrogel has adhesive properties towards living tissues on which it is disposed. The hydrogel can assist in the healing of annular tears that have occurred in a damaged vertebral disc. The hydrogel can also be biodegradable, dissolving over a period of time as healing progresses and normal tissue is laid down.

In an embodiment of the invention, an alkylated chitosan comprises a poly(oxyethylene)chitosan. A poly(oxyethylene)chitosan according to the present invention may have a degree of amino group substitution ranging down to about 0.1 (wherein only one in about every ten monomeric units is alkylated). A poly(oxyethylene)chitosan can bear a poly(oxyethylene) group of the chitosan amino group. Furthermore, a poly(oxyethylene)chitosan may also bear the poly(oxyethylene) derivative on one of the two free hydroxyl groups in a given monomeric unit, or may comprises a mixture of N- and O-alkylated chitosan monomeric units, or be di-alkylated or tri-alkylated on a single monomer unit. Thus, a fully alkylated chitosan monomeric unit has a degree of substitution of 3.0, and a poly(oxyethylene)chitosan according to the present invention may have a degree of substitution ranging up to 3.0 without departing from the principles of the invention. A preferred degree of substitution for a poly(oxyethylene)chitosan is about 0.35 to about 0.95. A particularly preferred degree of substitution is about 0.5. When the degree of substitution is less than about 1.0, it is believed that substitution is almost entirely on the chitosan amino group. An embodiment of a premix that forms a hydrogel adapted for practice of the inventive method includes a chitosan derivative, an alkylated chitosan. The degree of polymerization, that is, the number of monomeric units that make up the poly(oxyethylene)chitosan, may vary widely without departing from the principles of the invention. Any sample that contains more than a single molecule of the chitosan derivative will almost inevitably contain a distribution of molecules of different molecular weights, characterized by a weight-average molecular weight. A preferred poly(oxyethylene)chitosan according to the present invention has a weight-average molecular weight of about 200 kD to about 600 kD.

Another embodiment of a premix that forms a hydrogel according to the present invention comprises an acrylated chitosan. In a specific embodiment an alkylated chitosan comprises an acrylated chitosan wherein at least some of the free amino groups of the 2-amino-2-deoxyglycose monosaccharide monomeric units are substituted with acrylate groups. It is believed that acrylate groups are bonded to free amino groups of the chitosan via a Michael type conjugate addition wherein the nucleophilic amino group forms a bond to the β-carbon of the α,β-unsaturated acrylate, but the acrylate may be bonded to the chitosan in a different manner without departing from the principles of the invention. Furthermore, acrylates may themselves oligomerize after initial alkylation of the chitosan backbone. A preferred degree of substitution of the chitosan backbone with acrylate groups according to the present invention is about 0.25 to about 0.45.

The degree of polymerization, that is, the number of monomeric units that make up an acrylated chitosan according to the present invention may vary widely without departing from the principles of the invention. Any sample that contains more than a single molecule of an acrylated chitosan will almost inevitably contain a distribution of molecules of different molecular weights, characterized by a weight-average molecular weight. A preferred acrylated chitosan has a weight average molecular weight of about 200 kD to about 600 kD.

A premix also includes an oxidized polysaccharide. An oxidized polysaccharide, for example, an oxidized starch, an oxidized dextran, or an oxidized hyaluronan, can be prepared from the corresponding polysaccharide by oxidation, such as with sodium periodate, that brings about formation of aldehyde groups from the vicinal 2,3-diol units of the polysaccharides. Thus, a premix can include an acrylated chitosan and an oxidized polysaccharide, such as an oxidized dextran. The degree of oxidation of the polysaccharide refers to the number of monomeric units wherein the vicinal 2,3-diol unit has been cleaved to form a dialdehyde, in relation to the total number of monomeric units in the polymer. The degree of oxidation can be determined chemically, as is well known in the art and examples of which are provided below (e.g., Examples 3, 4, 6, and 7).

A premix can also include an alkylated chitosan, an oxidized polysaccharide, and an acidic polysaccharide. For example, a premix can include an acrylated chitosan, an oxidized dextran, and hyaluronic acid, in an aqueous medium. As a member of the class of acidic polysaccharides, a hyaluronan bears an ionizable carboxylic acid group on every other monosaccharide residue. The hyaluronan can be in the form of a hyaluronate, that is, with at least most of the carboxylic acid groups being in the ionized or salt form. Sodium hyaluronate is a specific example. The degree of substitution of carboxylic acid groups on the polymer backbone, assuming a monomeric unit comprising the disaccharide formed of one glucuronic acid monosaccharide and one 2-acetamido-2-deoxyglucose monosaccharide, is 1.0. Every monomeric unit (disaccharide unit) bears a single ionizable carboxylic acid group. A hyaluronan may be of any of a wide range of degrees of polymerization (molecular weights), but a preferred hyaluronan has a molecular weight of about 2,000 kD to about 3,000 kD.

In another embodiment, a premix that includes an acrylated chitosan and an oxidized polysaccharide, such as an oxidized dextran, can also include a carboxymethylcellulose. A carboxymethylcellulose is a derivative of cellulose (a β-1,4 linked polymer of glucose) wherein hydroxyl groups are substituted with carboxymethyl (—CH₂CO₂H) moieties. It is understood that the term carboxymethylcellulose comprises salts of carboxymethylcellulose, such as the sodium salt. A specific example of a premix comprises acrylated chitosan, carboxymethylcellulose sodium salt, a dehydrating reagent and a carboxyl activating reagent. Carboxymethylcellulose, as is well-known in the art, may have varying degrees of substitution, a “degree of substitution” referring to the number of derivatizing groups, herein carboxymethyl, per each monomer unit on the average. A particularly preferred carboxymethylcellulose according to the present invention has a degree of substitution of about 0.7 and a molecular weight of about 80 kD.

A premix according to the present invention comprises an aqueous medium. An aqueous medium necessarily includes water, and may include other components including salts, buffers, co-solvents, additional cross-linking reagents, emulsifiers, dispersants, electrolytes, or the like.

In another embodiment, the hydrogel contains therapeutic or protective agents that are released into the surrounding tissues on which the hydrogel is disposed within the vertebral disc. Examples of a therapeutic or protective agent that can be included in the hydrogel include an antibiotic, an anticancer agent, a peptide, a protein, a nucleic acid or a nucleic acid analog, a radioactive material, a recombinant protein, a pharmacologic agent, a plurality of stem cells, a plurality of exogenous stem cells, a growth factor, a blood product, or any combination thereof. Any of these agents can be included in the hydrogel by adding it to the premix prior to emplacement within the vertebral disc.

In another embodiment the hydrogel contains microspheres or nanospheres containing therapeutic agents or protective agents that further control the release of the agents from the hydrogel.

The inventive method provides that the premix is emplaced within an intervertebral disc in need thereof prior to gelation. This can be accomplished by any method known in the art for injection of liquid materials into spinal discs. For example, a syringe filled with freshly mixed premix can be inserted such that the tip resides within the nucleus pulposus of the target disc, then the substantially liquid premix injected in suitable volume. Emplacement of the premix can be observed using fluoroscopy or magnetic resonance imaging provided a suitable radiopaque or MRI-active agent has been included in the premix. Following injection of the premix, gelation occurs within minutes to provide the emplaced hydrogel.

The premix can be emplaced within the intervertebral disc using procedures well known to a person of skill in the art. For example: the skin over the vertebral disc space is anesthetized. Under fluoroscopy, a needle is inserted through the skin and soft tissues to the area above the pedicle and then into the disc space, lateral to the spinal cord and inferior to the nerve root. Contrast is injected and needle tip location is confirmed with imaging. The needle is advanced to the center of the disc space and a volume of prepared hydrogel is injected into the space. The hydrogel can contain a radio-opaque material or an MRI-active material to determine exact placement and to make sure there is no extravasation of hydrogel out of the disk space. After the bolus of hydrogel is given, the needle is removed. Multiple disk spaces can be treated in the same session.

### EXAMPLES

### Example 1

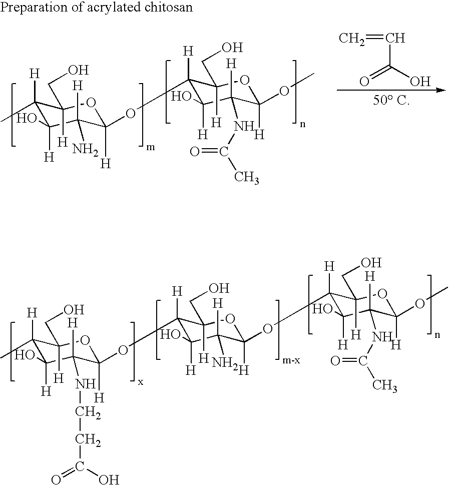

5.52 ml of acrylic acid was dissolved in 150 ml of double distilled water and 3 g of chitosan (Kraeber® 9012-76-4, molecular weight 200-600 kD) was added to it. The mixture was heated to 50 C. and vigorously stirred for 3 days. After removal of insoluble fragments by centrifugation, the product was collected and its pH was adjusted to 11 by adding NaOH solution. The mixture was dialyzed extensively to remove impurities.

### Example 2

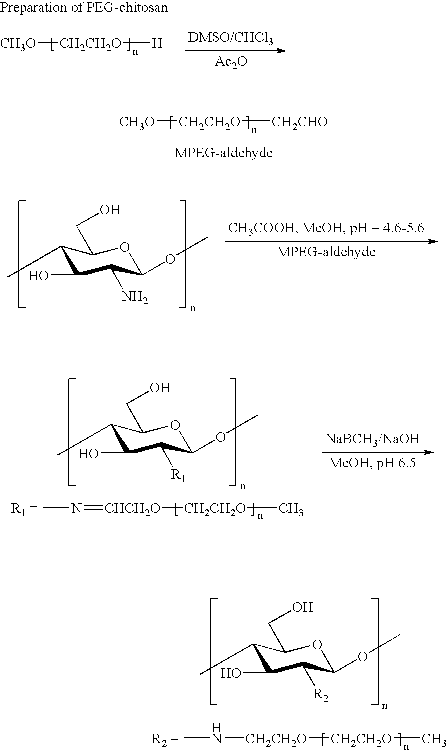

Monomethyl-PEG-aldehyde was prepared by the oxidation of Monomethyl-PEG (MPEG) with DMSO/acetic anhydride: 10 g of the dried MPEG was dissolved in anhydrous DMSO (30 ml) and chloroform (2 ml). Acetic anhydride (5 ml) was introduced into the solution and the mixture is stirred for 9 h at room temperature. The product was precipitated in 500 ml ethyl ether and filtered. Then the product was dissolved in chloroform and re-precipitated in ethyl ether twice and dried.

Chitosan (0.5 g, 3 mmol as monosaccharide residue containing 2.5 mmol amino groups, Kraeber 9012-76-4, molecular weight 200-600 kD) was dissolved in 2% aqueous acetic acid solution (20 ml) and methanol (10 ml). A 15 ml sample of MPEG-aldehyde (8 g, DC: 0.40) in aqueous solution was added into the chitosan solution and stirred for 1 h at room temperature. Then the pH of chitosan/MPEG-monoaldehyde solution was adjusted to 6.0-6.5 with aqueous 1 M NaOH solution and stirred for 2 h at room temperature. NaCNBH₃ (0.476 g, 7.6 mmol) in 7 ml water was added to the reaction mixture dropwise and the solution was stirred for 18 h at room temperature. The mixture was dialyzed with dialysis membrane (COMW 6000-8000) against aqueous 0.5 M NaOH solution and water alternately. When the pH of outer solution reached 7.5, the inner solution was centrifuged at 5,000 rpm for 20 min. The precipitate was removed. The supernatant was freeze-dried and washed with 100 ml acetone to get rid of unreacted MPEG. After vacuum drying, the final product (white powder) was obtained as water soluble or organic solvent soluble PEG-g-Chitosan. The yield of water soluble derivatives was around 90% based on the weight of starting chitosan and PEG-aldehyde.

### Example 3

### Preparation of Oxidized Dextran

Dextran (5 g) was dissolved in 400 mL of distilled H₂O, then 3.28 g of NaIO₄ dissolved in 100 mL ddH₂O was added. The mixture was stirred at 25° C. for 24 hrs. 10 ml of ethylene glycol was added to neutralize the unreacted periodate following by stirring at room temperature for an additional hour. The final product was dialyzed exhaustively for 3 days against doubly distilled H₂O, then lyophilized to obtain a sample of pure oxidized dextran.

### Example 4

### Analyses of Oxidized Dextran

The degree of oxidation of the oxidized dextran was determined by quantifying the aldehyde groups formed using t-butyl carbazate titration via carbazone formation. A solution of oxidized dextran (10 mg/ml in pH 5.2 acetate buffer) was prepared; and a 5-fold excess tert-butyl carbazate in the same buffer was added and allowed to react for 24 hrs at ambient temperature, then a 5-fold excess of NaBH₃CN was added. After 12 hrs, the reaction product was precipitated three times with acetone and the final precipitate was dialyzed thoroughly against water, followed by lyophilization. The degree of oxidation (i.e., abundance of aldehyde groups) was assessed using ¹H NMR by integrating the peaks: 7.9 ppm (proton attached to tert-butyl) and 4.9 ppm (anomeric proton of dextran).

### Example 5

### Preparation of an Oxidized Dextran/Acrylated Chitosan Gel

A 1 mL sample of a 1-3% aqueous oxidized dextran in water solution was mixed with 1 mL of a 1-3% aqueous acrylated chitosan solution. The mixture was gently stirred for 10 seconds. Gelation occurred within 30 seconds to 10 minutes at temperatures ranging from 5° C. to 37° C.

### Example 6

### Preparation of Oxidized Hyaluronan

Sodium hyaluronate (1.0 gram) was dissolved in 80 ml of water in a flask shaded by aluminum foil, and sodium periodate (various amounts) dissolved in 20 ml water was added dropwise to obtain oxidized hyaluronan (oHA) with different oxidation degrees. The reaction mixture was incubated at ambient temperature and 10 ml of ethylene glycol was added to neutralize the unreacted periodate following by stirring at room temperature for an additional hour. The solution containing the oxidized hyaluronan was dialyzed exhaustively for 3 days against water, then lyophilized to obtain pure product (yield: 50-67%).

### Example 7

### Analyses of Oxidized Hyaluronan

The degree of oxidation of oxidized hyaluronan was determined by quantifying aldehyde groups formed with t-butyl carbazate titration via carbazone formation. A solution of the oxidized hyaluronan (10 mg/ml in pH 5.2 acetate buffer) and a 5-fold excess tertbutyl carbazate in the same buffer were allowed to react for 24 hrs at ambient temperature, followed by the addition of a 5-fold excess of NaBH₃CN. After 12 hrs, the reaction product was precipitated three times with acetone and the final precipitate was dialyzed thoroughly against water, followed by lyophilization. The degree of oxidation (i.e., abundance of aldehyde groups) was assessed using ¹H NMR by integrating the peaks: 1.32 ppm (tert-butyl) and 1.9 ppm (CH₃ of hyaluronic acid).

What is claimed is:

1. A method of treatment of degenerative disc disease or of discogenic pain, comprising forming in situ within an intervertebral disc comprising a nucleus pulposus and an annulus fibrosus a biocompatible hydrogel, the hydrogel being formed by gelation of a substantially liquid premix, the hydrogel comprising an alkylated chitosan and an oxidized polysaccharide in an aqueous medium.

2. The method of claim 1 wherein the premix further comprises an acidic polysaccharide.

3. The method of claim 2 wherein the acidic polysaccharide comprises hyaluronan or carboxymethylcellulose.

4. The method of claim 1 wherein the alkylated chitosan comprises acrylated chitosan or poly(oxyalkylene)chitosan.

5. The method of claim 1 wherein the oxidized polysaccharide comprises oxidized dextran or oxidized starch.

6. The method of claim 1 wherein the premix is emplaced within the intervertebral disc prior to gelation.

7. The method of claim 6 wherein the premix is emplaced with a syringe or a catheter.

8. The method of claim 7 further comprising, prior to emplacing the premix, forming the premix using two mutually coupled syringes.

9. The method of claim 6 wherein the premix is emplaced within the nucleus pulposus and then flows into voids or tears in the annulus fibrosus prior to gelation.

10. The method of claim 6 wherein the premix is emplaced within voids or tears in the annulus fibrosus prior to gelation.

11. The method of claim 10 wherein the hydrogel seals or adhesively seals the voids or tears in the annulus fibrosus.

12. The method of claim 9 wherein the hydrogel seals or adhesively seals the voids or tears in the annulus fibrosus.

13. The method of claim 9 wherein the hydrogel fills a partially empty nucleus pulposus.

14. The method of claim 6 wherein the premix comprises a radiopaque agent or an MRI-active agent, and the premix is emplaced using fluoroscopy or MRI, respectively, for visualization.

15. The method of claim 1 wherein the hydrogel further comprises a therapeutic or protective agent.

16. The method of claim 15 wherein the therapeutic or protective agent comprises an antibiotic, an anticancer agent, a peptide, a protein, a recombinant protein, a nucleic acid or a nucleic acid analog, a radioactive material, a pharmacologic agent, a plurality of stem cells, a plurality of exogenous stem cells, a growth factor, a blood product, or any combination thereof.

17. The method of claim 16 wherein the pharmacologic agent comprises an anti-inflammatory agent.

18. The method of claim 15 wherein the pharmacologic agent comprises acetaminophen, indomethacin, a steroid, an interleukin, vascular endothelial growth factor, or a cytokine, or any combination thereof.

19. The method of claim 15 wherein the therapeutic or protective agent is contained within a microsphere or a nanosphere disposed in the hydrogel.

20. The method of claim 15 wherein the therapeutic or protective agent is added to the premix prior to emplacing the premix within in the vertebral disc.

21. The method of claim 1 wherein the hydrogel is biodegradable.

22. The method of claim 1 wherein the hydrogel induces tissue growth within the vertebral disc.

23. A kit for carrying out the method of claim 6, comprising a first container comprising an alkylated chitosan, a second container comprising an oxidized polysaccharide, wherein each container respectively contains an aqueous medium or is adapted for addition of an aqueous medium thereto; a container for mixing the contents of the first container and the second container in an aqueous medium to provide the premix; a syringe or pump for transferring the premix; a tube or catheter or needle for conducting the premix to a locus within a target vertebral disc; and, optionally, instructional materials.

24. The kit of claim 23 wherein an acidic polysaccharide is contained within the first container, the second container, or both.

25. The kit of claim 23 wherein the container for mixing is the first container or the second container.

26. The kit of claim 23 wherein the first container, the second container, or both further comprises a radiopaque or an MRI-active material.

27. The kit of claim 23 wherein the alkylated chitosan is acrylated chitosan or poly(oxyalkylene)chitosan.

28. The kit of claim 23 wherein the oxidized polysaccharide is oxidized dextran, oxidized starch, or oxidized hyaluronan.

29. The kit of claim 24 wherein the acidic polysaccharide is hyaluronan, oxidized hyaluronan, or carboxymethyl cellulose.

30. The kit of claim 23 wherein the alkylated chitosan is acrylated chitosan, the acidic polysaccharide is hyaluronic acid, and the oxidized polysaccharide is oxidized dextran.

31. The kit of claim 23 wherein the first or the second container or both respectively comprises a syringe.

32. The kit of claim 31 wherein the contents of the first container and the second container are mixed at least in part in the syringe to provide a premix-charged syringe.

33. The kit of claim 32 wherein the premix-charged syringe is adapted to dispose the premix within the intervertebral disc through a needle or a catheter.
